(19) United States
(12) Patent Application Publication
(10) Pub. No.: US 2008/0319349 A1
(43) Pub. Date: Dec. 25, 2008

(54) System and Related Method For Determining a Measurement Between Locations on a Body

(76) Inventor: Yitzhak Zilberman, Valencia, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/911,882

(22) PCT Filed: Apr. 17, 2006
(86) PCT No.: PCT/US06/14455

§ 371 Date: Jun. 25, 2008

Related U.S. Application Data
(60) Provisional application No. 60/672,915, filed on Apr. 18, 2005.

(51) Int. Cl. A61B 5/103

(52) U.S. Cl. 600/587

(57) ABSTRACT

A system and related method for characterizing an effect of a rehabilitation therapy on a body. The apparatus includes a first device, which is configured to be coupled to the body at a first location, and a second device, which is configured to be coupled to the body at a second location that is separated from the first location by a distance. The first device is configured to generate a wireless signal. The second device is configured to detect the wireless signal and to generate data based on the detected wireless signal that is configured to be used to calculate the distance. The distance is used to characterize the effect of the rehabilitation therapy on the body.

### BACKGROUND OF THE INVENTION

1. Field of the Invention

The invention relates generally to the field of systems that are used for determining a measurement between locations on a body. More specifically, the invention relates to a system and related method for measuring the distance and/or the orientation between locations on a body, and characterizing an effect of a rehabilitation therapy on the body based on the measurement.

2. Description of the Related Art

Stroke is a leading cause of permanent impairment and disability. For example, approximately 70 percent of all stroke survivors have a paralyzed limb, e.g., an arm or a hand. Stroke victims that receive rehabilitative therapy soon after the stroke, typically within the first three months after a stroke, may recover some of the original mobility of their impaired limb(s). Several techniques have been developed to help make the rehabilitation process for stroke victims more efficient, and to aid in the assessment of the patient's progress. Some of these techniques include manual rehabilitation performed by a therapist, using simple rehabilitation tools. The therapist can assess the patient's progress during the rehabilitation process using a variety of methods, including, for example, the Stroke Rehabilitation Assessment of Movement (“STREAM”) test, which associates scores on the Box and Block Test, the Balance Scale, and the Barthel Index, which are known to those having ordinary skill in the art.

A patient performs the Box and Block test using a box that includes a partition, which divides the box into two equal compartments. A number of small wooden blocks are placed one of the box's compartments. During the test, the patient is required to use the affected limb, e.g., the arm and hand that are impaired due to the stroke, to move as many blocks as possible from one of the box's compartments to the other compartment in 60 seconds. The patient can move the blocks only by grasping one block at a time, transporting the block over the partition, and releasing the block into the other compartment. Once the test is complete, the number of blocks transported from one compartment to the other compartment is counted. Some other devices that are used for the assessment of spasticity are, for example, the BIODEX MULTI-JOINT SYSTEM II isokinetic dynamometer, which is available from Biodex Medical System of Shirley, N.Y.; and the RIGIDITY ANALYZER by Prochazka of Edmonton, Canada.

An alternative to having a therapist manually perform rehabilitation therapy on a stroke victim is to use a robotic rehabilitation device. Robotic rehabilitation devices can combine both training and assessment capabilities in the same device. For example, the robot can cause the patient to move his or her impaired limb according to a preferred trajectory, or to access the patient's progress in voluntarily tracking a cursor on a screen with the impaired limb. Some of the robots that are available on the market are offered by Interactive Motion Technologies, Inc. (“IMT”) of Cambridge, Mass.; and Rehab Robotics Limited, Staffordshire University of Staffordshire, United Kingdom.

An example of a device that recently has been used to measure the mobility of a patient's impaired limb is an angle measurement device called a goniometer. Several types of goniometers are known in the art. Example goniometers can determine angle measurements based on changes in the resistance of a fluid in a tube as the tube is bent, changes in the optical properties of an optical fiber as the optical fiber is bent, the rotation of wheels, and/or the extension of cables. However, these goniometers typically require a physical interconnection, for example, via a tube, a fiber, wires, and/or cables, between the points on the patient's body that are to be compared during the angle measurement. An example goniometer is the MLTS700 JOINT ANGLE SENSOR by PowerLab of New South Wales, Australia. Additional examples of goniometers are discussed in U.S. Patent Application Publication Number 2003/0083596 to Kramer et al. and U.S. Pat. No. 6,651,352 to McGorry et al.

Recently, virtual reality applications have boosted various types of 3-D tracking and positioning devices for wrists and fingers, for example the CYBERGLOVE by Immersion Corporation of San Jose, Calif. The CYBERGLOVE is available in an eighteen sensor model, which features two bend sensors on each finger, four abduction sensors, and sensors for measuring thumb crossover, palm arch, wrist flexion, and wrist abduction. The CYBERGLOVE also is available in a twenty-two sensor model, which includes additional sensors that are used to measure the flexion and wrist abduction

The devices discussed above and the currently available tools that are used to assess the mobility of a patient's limb and the patient's progress during rehabilitation therapy are considered to be poor proxies for the everyday use of an impaired limb. Also, many of the currently available tools require a therapist to play an active role during the assessment procedure. Accordingly, there is a need for a system that is configured to assess the mobility of a stroke patient's impaired limb(s) during rehabilitation therapy, which can include the patient's everyday use of the limb(s) and physical therapy. The present invention satisfies this need, as well as other needs as discussed below.

### SUMMARY OF THE INVENTION

The invention resides in a system and a related method for assessing the mobility of a stroke patient's impaired limb(s) during rehabilitation therapy, including everyday use. An exemplary embodiment of the present invention is a system that is configured to characterize an effect of a rehabilitation therapy on a body. The system includes a first device, which is configured to be coupled to a body at a first location, and a second device, which is configured to be coupled to the body at a second location that is separated from the first location by a first distance. The first device is configured to generate a first wireless signal. The second device is configured to detect the first wireless signal and to generate data based on the detected first wireless signal that is configured to be used to calculate the first distance. The first distance is used to characterize the effect of the rehabilitation therapy on the body.

In other, more detailed features of the invention, the system further includes a third device, which is configured to be coupled to the body at a third location that is separated from the second location by a second distance. The first device is configured to generate the first wireless signal at a first frequency. The third device is configured to generate a second wireless signal at a second frequency. The second device is configured to detect the second wireless signal and to generate additional data based on the detected second wireless signal. The additional data is configured to be used to calculate the second distance, which is used to characterize the effect of the rehabilitation therapy on the body. Also, the third device can be configured to generate the second wireless signal at the same time that the first device is configured to generate the first wireless signal. In addition, the second device can be configured to detect in a selectable manner the first wireless signal or the second wireless signal.

In other, more detailed features of the invention, the apparatus further includes an external device, which is configured to communicate with the second device. The second device is configured to communicate the data to the external device. The external device is configured to calculate the first distance based on the data. Also, the external device can be configured to calculate an angle of orientation between the second device and the first device based on the data. In addition, the external device can be configured to communicate with the second device via a wireless communication path that is a radio frequency path, an electrical current path through the body, a path configured for the communication of modulated sonic waves, a path configured for the communication of modulated ultrasonic waves, and/or an optical communication path.

In other, more detailed features of the invention, the external device is configured to calculate one or more of the following values: an average of the first distance over a period of time, a standard deviation of the first distance over a period of time, a number of times that the second device is moved relative to the first device over a period of time based on the first distance, a velocity of the second device relative to the first device based on the first distance, an average velocity of the second device relative to the first device over a period of time based on the first distance, an acceleration of the second device relative to the first device based on the first distance, and an average acceleration of the second device relative to the first device over a period of time based on the first distance.

In other, more detailed features of the invention, the first device and/or the second device is configured to be implanted into the body, or attached to the body using an adhesive, a piece of clothing, a strap, a belt, a clip, and/or a watch. Also, the first device can be coupled to the torso of the body, and the second device can be coupled to a hand or an arm of the body. In addition, the wireless signal can be a magnetic field, a low-frequency magnetic field, a sonic wave, or an ultrasonic wave.

In other, more detailed features of the invention, the first device and/or the second device includes a component that is a battery, a coil, orthogonal coils, a generator, a voltage measurement circuit, a transducer, a processing circuit, a transmitter, a receiver, and/or a transceiver. Also, the first device and/or the second device can be a miniature stimulator. In addition, the first device and/or the second device can include a transmitter and a receiver.

Another exemplary embodiment of the present invention is a system that is configured to characterize an effect of a rehabilitation therapy on a body. The system includes a transmitter, a plurality of receivers, and an external device. The transmitter is configured to be coupled to a body at a first location, and each of the plurality of receivers is configured to be coupled to the body at a different location that is separated from the transmitter by one of a plurality of distances. The external device is configured to communicate with the plurality of receivers. The transmitter is configured to transmit a wireless signal. Each of the plurality of receivers is configured to detect the wireless signal, to generate data based on the detected wireless signal, and to communicate the data to the external device. The external device is configured to calculate the plurality of distances between the plurality of receivers and the transmitter based on the data. The plurality of distances is used to characterize the effect of the rehabilitation therapy on the body.

In other, more detailed features of the invention, the wireless signal is an ultrasonic wave, and the external device is configured to calculate the plurality of distances based on an amplitude of the ultrasonic wave detected by each of the plurality of receivers, a phase of the ultrasonic wave detected by each of the plurality of receivers, and/or a time of propagation of the ultrasonic wave to each of the plurality of receivers.

In other, more detailed features of the invention, the external device is configured to calculate a plurality of angles of orientation between the plurality of receivers and the transmitter based on the data. Also, the system can further include an additional device that is coupled to the external device and configured to aid in the calculation of the plurality of distances and the plurality of angles of orientation. The additional device can be a distance sensor, an angle sensor, an acceleration sensor, a vibration sensor, and/or a video camera. In addition, the external device can be configured to calculate, based on the data, a velocity of each of the plurality of receivers relative to the transmitter, and/or an acceleration of each of the plurality of receivers relative to the transmitter.

In other, more detailed features of the invention, the body includes a healthy limb and a corresponding impaired limb. One of the plurality of receivers is configured to be coupled to the healthy limb, and another of the plurality of receivers is configured to be coupled to the impaired limb. The external device is configured to compare the distance between the one of the plurality of receivers and the transmitter to the distance between the another of the plurality of receivers and the transmitter.

An exemplary method according to the invention is a method for characterizing an effect of a rehabilitation therapy on a body. The method includes providing a first device that is configured to be coupled to the body at a first location and configured to transmit a wireless signal, providing a second device that is configured to be coupled to the body at a second location and configured to detect the wireless signal, using the first device to transmit the wireless signal, using the second device to detect the wireless signal, calculating a distance between the first device and the second device based on the wireless signal that is detected by the second device, and using the distance to characterize the effect of the rehabilitation therapy on the body.

Other features of the invention should become apparent from the following description of the preferred embodiments taken in conjunction with the accompanying drawings, which illustrate, by way of example, the principles of the invention.

### BRIEF DESCRIPTION OF THE DRAWINGS

FIGS. 1A and 1B are illustrations of a system according to an embodiment of the present invention that is configured to monitor the range of movement of a patient's arm and hand relative to his or her torso.

FIGS. 2A and 2B are illustrations of another system according to an embodiment of the present invention that is configured to monitor the range of movement of a patient's arm and hand relative to his or her torso.

FIG. 3 is a perspective illustration of a battery powered miniature stimulator.

FIG. 4 is a block diagram of a system according to an embodiment of the present invention that includes battery powered miniature stimulators.

FIG. 5 is a block diagram of another system according to an embodiment of the present invention that includes battery powered miniature stimulators.

FIG. 6 is an illustration of a system according to an embodiment of the present invention that is configured to measure the distance and the orientation between a transmitter and a receiver.

FIG. 7 is an illustration of a system according to an embodiment of the present invention that is configured to measure the distance between a transmitter and a receiver.

FIG. 8 is an illustration of a system according to an embodiment of the present invention that is configured to measure the distance between an ultrasonic transmitter and an ultrasonic receiver.

FIGS. 9A, 9B, and 9C are illustrations of signal characteristics including signal amplitude, phase difference, and time of arrival difference, respectively, for an ultrasonic signal.

FIG. 10 is a schematic illustration of a data acquisition system according to an embodiment of the present invention.

FIG. 11 is a flow diagram of an exemplary algorithm according to the present invention.

FIG. 12 is a graph of a daily average distance between a patient's healthy hand and his or her torso as a function of time, and a daily average distance between the patient's stroke-affected hand and his or her torso as function of time.

### DETAILED DESCRIPTION OF THE PREFERRED EMBODIMENTS

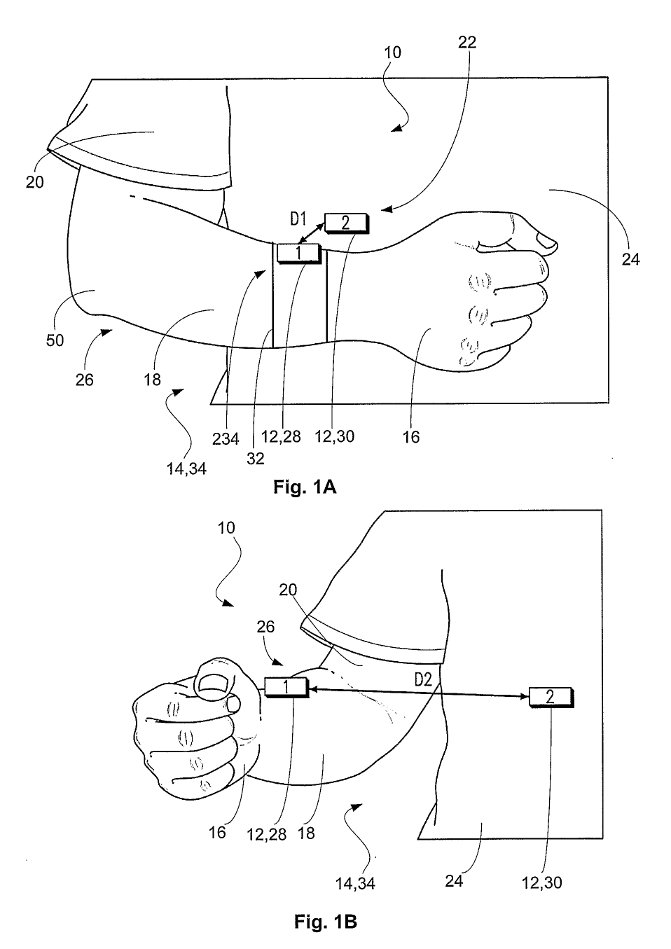

Embodiments of the present invention provide for a relatively inexpensive and portable approach for assessing the mobility of a patient's limb(s) during rehabilitation therapy, including routine daily activity. Referring to FIGS. 1A-B, embodiments of the present invention are systems 10 that include miniature devices 12, which are configured to communicate with one another wirelessly, and do not require any physical interconnection between the devices. Embodiments of the present invention utilize these miniature devices to measure static and motion parameters for parts of a patient's body 14.

In specific embodiments, the devices 12 are used to measure the following: the distance and/or angle between the patient's hand 16, forearm 18, and/or upper arm 20 and a predetermined location 22 on the patient's body 14, e.g., the patient's torso 24; the angle between the patient's hand and forearm; and the speed and/or the acceleration of the patient's hand and arm relative to the predetermined location. By measuring the distance, angle, and/or the motion parameters between two or more locations on the patient's body, the mobility and rehabilitation status of parts of the body, e.g., the hand and the arm, can be assessed and tracked.

By tracking the distance between locations on the patient's body, the maximal and typical values of displacement of a part 16-20 of the patient's body 14 can be calculated during daily activity. These displacement values can be used to evaluate the effectiveness of rehabilitation therapy, including everyday use, on the patient's body. In extreme disability cases, an impaired limb, e.g., the patient's arm 26, will be kept in close proximity to the patient's torso 24, and with a limited amount and range of movement. As a result of the rehabilitation process, it is expected that the amount and range of the movement of the impaired limb will increase over time.

In FIGS. 1A-B, the miniature devices 12, i.e., a first device 28 and a second device 30, are coupled to a patient's forearm 18 and torso 24, respectively. One or both of the devices can be coupled to the patient's body 14 by attaching the device(s) to the patient using adhesive and/or straps 32, or implanting the device(s) into the patient's forearm and/or torso. FIG. 1A shows the patient 34 holding his or her arm 26 in close proximity to his or her torso, in such a manner that the first device and the second device are near one another. Thus, in FIG. 1A, the distance D1 between the first device and the second device is relatively short. In the configuration shown in FIG. 1B, the patient's arm is positioned away from his or her torso, and the distance D2 between first device and second device is greater than D1.

By measuring the distance between the first device 28 and the second device 30 at different times, the range of arm movement by the patient 34 can be measured. The distance measurement data can be stored in a memory (not shown) in the devices, and later transmitted to an external device (discussed below) for analysis of the data. In the embodiment of FIGS. 1A-B, the first device can be a transmitter of a wireless signal and second device can be a receiver that is configured to detect the wireless signal, or vice versa. A wireless signal is a detectable physical quantity, for example, a field, e.g., an electric field or a magnetic field, or a wave, e.g., a sonic wave or an ultrasonic wave, that is propagated between two points in space without the use of electrical wires. By measuring the distance and/or the orientation of the first device relative to the second device, it is possible to calculate the position and orientation of the patient's arm 26 relative to his or her torso 24.

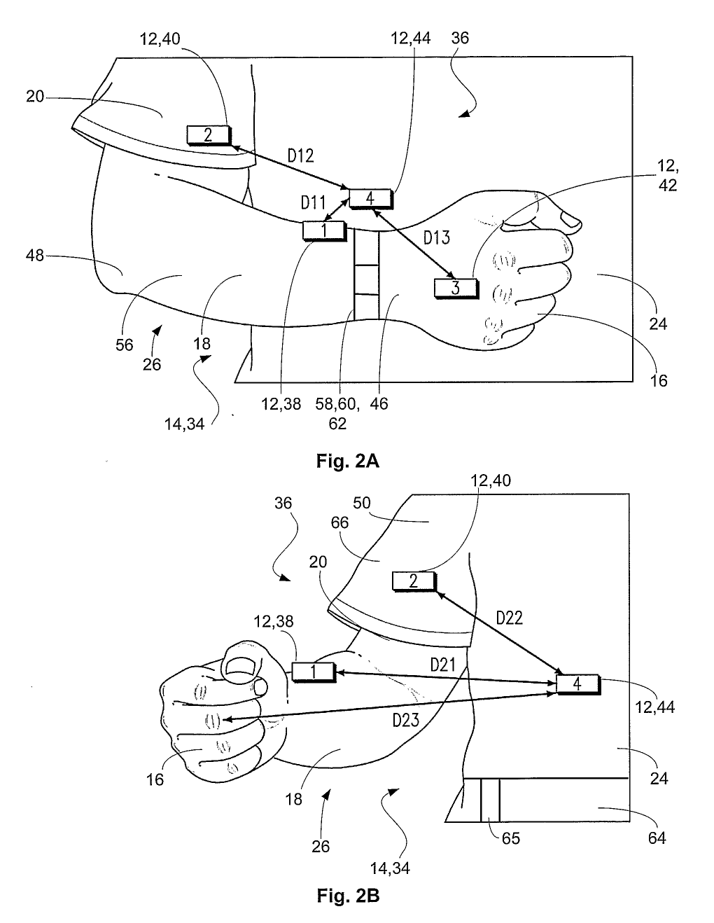

More than a single pair of miniature devices 12 can be attached to and/or implanted in the patient 34. An example embodiment of a system 36 that includes more than a single pair of devices is illustrated in FIGS. 2A-B, where the patient has a plurality of devices, specifically four devices 38-44, coupled to, e.g., attached to and/or implanted in, his or her body 14. The term “plurality,” as used in this document, can mean two or more.

In FIGS. 2A-B the first device 38 is coupled to the patient's forearm 18; the second device 40 is coupled to the patient's upper arm 20; the third device 42 is coupled to the patient's hand 16; and the fourth device 44 is coupled to the patient's torso 24. In the embodiment shown in FIGS. 2A-B, the fourth device can be a transmitter and the first, second, and third devices can be receivers. By measuring the distance and orientation between each of the receivers relative to the transmitter, it is possible to use the system 36 to calculate the position, orientation, and movement of the patient's hand, forearm, and upper arm relative to the patient's torso.

In another embodiment, the first device 38, the second device 40, and the third device 42 are transmitters and the fourth device 44 is a receiver. In this embodiment, each of the first device, the second device, and the third device transmits a wireless signal at a unique frequency. Thus, the wireless signal output from the first device has a frequency that is different from the frequencies of the wireless signals output from the second device and the third device. Two or more of the first, second, and third devices can transmit simultaneously their respective wireless signals, or the wireless signals can be transmitted at different times. The fourth device is configured to receive the wireless signals output from the first, second, and third devices in a selectable manner. Thus, the fourth device can be tuned to receive just one of the three wireless signals. By tuning to the frequency of the wireless signal output from one of the first, second, and third devices, the fourth device can receive the wireless signal from that device, and can use the received wireless signal to generate data that is used to calculate the position, orientation, and/or movement of that device relative to the fourth device.

In the embodiments of FIGS. 2A-B, even though the system 36 includes four devices 12 that are configured as three receivers and one transmitter, or three transmitter and one receiver, it should be understood that, in additional embodiments, the system can include both a plurality of transmitters and a plurality of receivers. In other embodiments, the system can include a device having both a transmitter and a receiver, and thus, the device can function in either capacity.

In FIGS. 2A-B, a plurality of distances D1-D23 is shown, three distances D11-D13 in FIG. 2A and three distances D21-D23 in FIG. 2B. FIG. 2A is similar to FIG. 1A in that it shows the patient's arm 26 positioned close to his or her torso 24. In FIG. 2A, the distance D11 between the first device 38 and the fourth device 44; the distance D12 between the second device 40 and the fourth device; and the distance D13 between the third device 42 and the fourth device are relatively short in comparison to the respective distances, D21, D22, and D23, in FIG. 2B where the patient's arm is extended away from his or her torso. In addition to using the devices 12 to measure the distance of movement for a limb, in other embodiments, measurements of the angles between the devices can be performed. Thus, the system 36 shown in FIGS. 2A-B also allows for orientation and movement measurements for the patient's wrist 46, elbow 48, and shoulder 50.

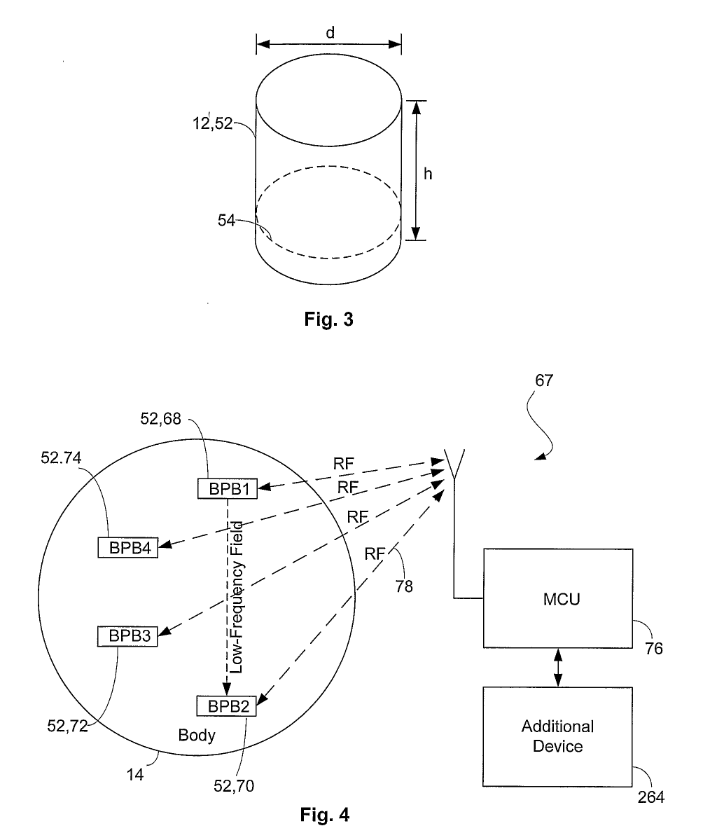

Referring additionally to FIG. 3, each of the miniature devices 12 can be a miniature, implantable, battery powered stimulator, for example, a Battery Powered BION (“BPB”) 52, which includes a miniature, rechargeable battery 54 and is made by Advanced Bionics Inc. of Santa Clarita, Calif. Each BPB is generally cylindrical in shape and has a diameter d of approximately 3 mm and a height h of approximately 25 mm. As previously discussed, these devices can be implanted in the patient's body 14 or attached externally to the patient's skin 56 using straps 32 or an adhesive, e.g., an adhesive band (see FIGS. 1A and 2A). Also, the devices can be embedded in consumer devices 58 that are positioned on or near the patient's skin using a holder or coupler 60, e.g., a watch 62, a belt 64, or a clip 65 (see FIGS. 2A-B). Also, the devices can be attached to a piece of the patient's clothing 66, if the piece of clothing is tight enough to the body to follow the movement of parts 16, 18, and 20 of the patient's body.

Each of the BPB 52 can be programmed to operate as a transmitter or a receiver. In particular, each BPB can include the ability to do the following: to deliver electrical stimulation, to generate ultrasonic signals, to measure biopotentials, to transmit and receive low-frequency magnetic field, and to transmit and receive bi-directional radio frequency (“RF”) telemetry to/from an external device (discussed below). An example embodiment of a BPB is discussed in Schulman J., et al., “Battery Powered BION FES Network,” 2005—Electronics, IEEE-EMBS, Transaction of 26th IEEE EMBC Meeting, p. 418, September, 2004, which is incorporated by reference herein.

In the embodiment of FIGS. 1A-B, it is likely that the second device 30 is a transmitter and the first device 28 is a receiver; while in FIGS. 2A-B, it is likely that the fourth device 44 is a transmitter and the first, second, and third devices 38, 40, and 42, respectively, are receivers, because, typically, a transmitter is located on or in the torso 24 rather than on or in the hand 16 or arm 26. The reason being, is that the transmitter usually is larger than the receiver because it includes larger components and larger batteries 54, which can be used to generate the low-frequency magnetic fields (discussed in greater detail below).

FIG. 4 is a block diagram of a system 67 according to an embodiment of the present invention that includes a plurality of BPBs 52, e.g., BPB1 68, BPB2 70, BPB3 72, and BPB4 74. The BPBs are all coupled to, e.g., attached to and/or implanted in, a patient's body 14 at different locations. An external device 76, e.g., a master control unit (“MCU”), is configured to maintain wireless communication, e.g., radio frequency (“RF”) communication 78, with each of the implanted BPBs. Various RF bands can be used for communication between the MCU and the BPBs, including the UHF band.

During use, the MCU 76 is configured to send commands and data to the BPBs 52, for example, to start or stop stimulation and/or to change the stimulation parameters. The BPBs are configured to send data, e.g., status information and measurement data, back to the MCU. In one embodiment, as shown in FIG. 4, BPB1 68 is configured to generate a wireless signal, e.g., a low-frequency magnetic field, which is detected and measured by BPB2 70. After processing the signal, BPB2 communicates the results of its measurements to the MCU, which is configured to calculate the distance between BPB2 and BPB1 based on the data communicated from BPB2.

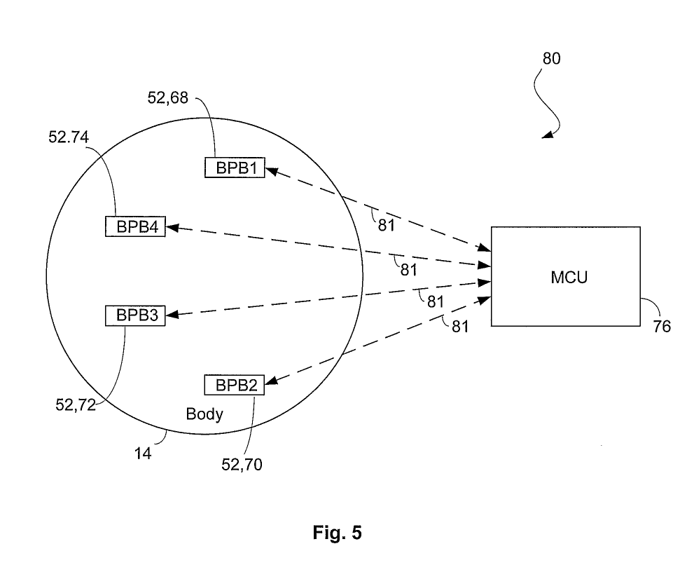

Referring additionally to FIG. 5, in additional example embodiment systems, the BPBs 52 are configured to communicate with the MCU 76 using a communication path 81 other than an RF communication path. For example, the tissue of the body that surrounds a BPB can be used as a communication path. In this example embodiment, the BPB that is operating as a transmitter can transmit modulated, low-amplitude, electrical current into the body instead of transmitting RF telemetry, or in addition to transmitting RF telemetry. In this example, the MCU is configured to detect and demodulate the electrical current that has been transmitted through the patient's body. The MCU can be coupled to the body to facilitate the receipt of the transmitted electrical signal. In additional example embodiments, the BPBs are configured to communicate with the MCU using a path 81 that is configured for the communication of modulated sonic waves, modulated ultrasonic waves, and/or optical signals, e.g., infrared signals.

As was the case in the embodiments of FIGS. 1A-B and 2A-B, by calculating the distance between the devices 12 and 52, the amount and range of movement between the devices can be determined. In the systems 67 and 80 shown in FIGS. 4 and 5, the calculated distances between the devices can be stored in the MCU 76 for later analysis.

Distance and Orientation Determined from Low-Frequency Magnetic Field Measurements:

In embodiments of the present invention, distance and/or orientation measurements are determined based on a magnetic field that is generated by one of the devices 12, e.g., a transmitter, and detected by another device, e.g., a receiver. The magnetic field can be, for example, a low-frequency magnetic field, i.e., a magnetic field having a frequency from less than approximately 10 KHz to several hundred KHz. If orthogonal low-frequency magnetic fields are utilized, the distance and orientation of the receiver relative to the transmitter can be calculated. This approach usually requires the use of three miniature orthogonal coils in both the transmitter and the receiver.

Examples of systems that include three orthogonal coils in the transmitter and the receiver are the MEDICAL POSITIONING SYSTEMS by Medical Guidance Systems (“Mediguide”) of Israel, which are used for intra-body navigation of catheters (see U.S. Pat. No. 6,233,476 to Strommer and Eichler). In the MEDICAL POSITIONING SYSTEMS, transmitting coils are located in a bed on which the patient 34 rests, and miniature receiving coils are embedded in the tip of a catheter that is to be inserted into the patient. During insertion of the catheter, the receiving coils are used to detect the position and orientation of the catheter relative to the transmitting coils in the bed.

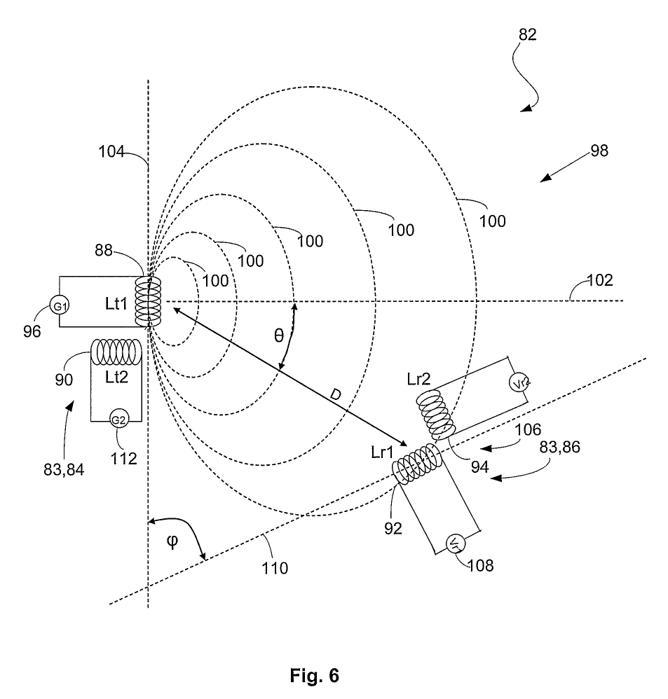

FIG. 6 is an illustration of a system 82 that includes two devices 83, i.e., a transmitter 84 and a receiver 86, according to an embodiment of the present invention. FIG. 6 will be referenced in the following discussion of the principles of operation for embodiments of the present invention when using a low-frequency magnetic field to determined distance and angle of orientation. While the devices that are used in the embodiments of the present invention can include three orthogonal coils, for the sake of simplicity, FIG. 6 is limited to 2-D space, and thus, only shows two 88 and 90 of the three orthogonal coils for the transmitter and two 92 and 94 of the three orthogonal coils for the receiver.

The transmitter 84 includes a transmitting coil Lt1 88, which is coupled to and driven by a generator G1 96. Lt1 generates a magnetic field M1 98, which is proportional to G1's output. Lines of magnetic field for M1 are shown as curved dashed lines 100 in FIG. 6. The value of the magnetic field output by Lt1 and detected by the receiver 86 depends on the distance D between Lt1 and the receiver, and the angle θ between a perpendicular 102 to Lt1's axis 104 and the receiver's location 106. Thus, the value of M1 detected by the receiver is a function of D and θ.

The receiver 86, which is configured to detect the magnetic field 98, includes a first receiving coil Lr1 92. The magnetic field induces a voltage Vr1 108 in Lr1. The value of Vr1 depends on the following: Lr1's coil geometry, e.g., the length of the coil, the diameter of the coil, and the number of turns of the coil; the intensity of M1; and the angle φ between Lt1's axis 104 and Lr1's axis 110. The following is a mathematical expression for Vr1 as a function of G1, which is denoted Vr11:

Vr11=f11(G1,D,θ,φ), where D, θ, and φ are unknown.

The unknown values can be calculated by inserting additional coils 90 and 94. For example, the transmitter 84 can include another transmitting coil Lt2 90, which is orthogonal to Lt1 88. Also, the receiver 86 can include another receiving coil Lr2 94, which is orthogonal to Lr1 92. Assuming that the pairs of orthogonal coils, Lt1 and Lt2, and Lr1 and Lr2, are small and positioned close to one another, it can be assumed that the same distance D and the same angle θ can be used in all of the calculations.

During use, the transmitting coils Lt1 88 and Lt2 90 can be operated in turn, or operated at different frequencies, to distinguish between the voltages induced in Lr1 92 and Lr2 94. The following are corresponding equations for the voltage Vr12, which is induced in receiving coil Lr1 as a function of the magnetic field (not shown) generated by G2 112, the voltage V21, which is induced in receiving coil Lr2 as a function of the magnetic field 98 generated by G1 96, and the voltage V22, which is induced in receiving coil Lr2 as a function of the magnetic field generated by G2:

Vr12=f12(G2,D,θ,φ),

Vr21=f21(G1,D,θ,φ), and

Vr22=f22(G2,D,θ,φ).

The three unknown values D, θ, φ can be calculated using the above equations for V11, V12, V21, and V22, resulting in the relative distance and angle of orientation of Lr1 and Lr2 relative to Lt1 and Lt2. Similar calculations can be applied to systems that include a transmitter 84 and a plurality of receivers 86, thus, resulting in a plurality of distances D and a plurality of angles of orientation φ.

One having ordinary skill in the art should understand that in a 3-D scenario, the transmitter 84 includes a third transmitting coil Lt3 (not shown) and generator G3 (not shown), and the receiver 86 includes a third receiving coil Lr3 (not shown) and induced voltage Vr3. The distance and orientation angle of all of coils 88-94 in the 3-D scenario are determined in an analogous manner to that previously described for the 2-D scenario.

Distance Determined from Low-Frequency Magnetic Field Measurements:

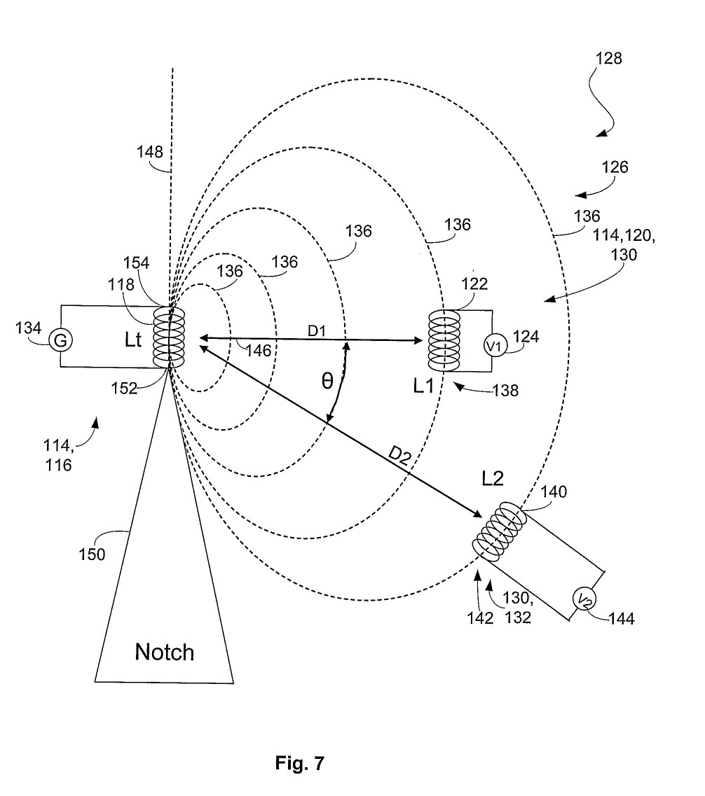

When tracking the mobility of a part 16-20 and 26 of the patient's body 14, e.g., the patient's hand 16 or arm 26, there may be a need to only measure the distance of movement, and not the orientation. When this is the case, referring to FIG. 7, it is possible to measure the distance that the part of the patient's body moves by measuring the distance between two devices 114, i.e., a transmitter 116 having a transmitting coil 118 and a receiver 120 having a receiving coil 122. This can be done by measuring a voltage 124 that is induced in the receiving coil by a magnetic field 126, for example, a low-frequency magnetic field that is generated by the transmitting coil.

FIG. 7 illustrates a system 128 for measuring distance using low-frequency magnetic field 126. The system includes the transmitter 116 and a plurality of receivers 130, which includes a first receiver 120 and a second receiver 132. The first receiver and the second receiver can be coupled to different locations on the patient's body 14. The transmitter includes a low-frequency generator G 134, which supplies current to a transmitting coil Lt 118. Lt generates a magnetic field, which spreads out into three-dimensional space. Lines of magnetic field are shown as curved dashed lines 136 in FIG. 7. The magnitude of the magnetic field usually decreases according to the cubic power of distance from Lt.

The first receiver 120 includes a first receiving coil L1 122 that is configured to detect the magnetic field 126 generated by the transmitter 116, which induces a voltage V1 124 in L1. V1 is dependent upon the magnitude of the magnetic field at L1's location 138, and L1's physical parameters, e.g., the length of L1, the diameter of L1, and the number of turns of L1. Similarly, the second receiver 132 includes a second receiving coil L2 140, which is configured to detect the magnetic field generated by the transmitter, and the detected magnetic field at L2's location 142 will induce a voltage V2 144 in L2.

Keeping all of L1's and L2's physical parameters the same, the values of V1 124 and V2 144 will be dependent on the distance D1 between L1 122 and Lt 118, and the distance D2 between L2 140 and Lt, respectively. It is possible to correlate V1 to D1 and V2 to D2, and the resulting correlations can be formalized into a calibration table (not shown). The correlations between V1 and D1, and V2 and D2 are almost totally independent of the angle θ, which is the angle between a receiving coil's position, e.g., L1's or L2's position 138 or 142, respectively, relative to a perpendicular 146 to Lt's axis 148.

Therefore, the plurality of distances, D1 and D2, between Lt 118 and L1 122, and Lt and L2 140, respectively, can be calculated by measuring V1 124 and V2 144, respectively. It should be noted that the assumption about the independence of the measured voltage, e.g., V1 and V2, from the angle θ is not valid for the narrow range of angles 150 that is identified as the notch in FIG. 7. While the notch is shown only at one end 152 of Lt in FIG. 7, one having ordinary skill in the art should understand that a mirror image of the notch also exists at the opposite end 154 of Lt. Experimentally, it has been demonstrated that by using this technique distances of up to 20 cm can be measured using a 127 KHz magnetic field. Greater distances can be measured by increasing the transmission power of G 134.

Distance Determined from Sonic or Ultrasonic Measurements:

In other embodiments, the distance between devices 12 can be measured based on the amplitude, phase, and/or time of propagation of a sonic wave(s), i.e., a wave(s) having a frequency from approximately 20 Hz to approximately 20 KHz, or ultrasonic wave(s), i.e., a wave(s) having a frequency from approximately 20 KHz to approximately 10 MHz. Referring again to FIG. 1A, sonic and ultrasonic wave(s) are types of wireless signals that can be transmitted from a transmitter 30 to a receiver 28. Ultrasonic distance measurement devices are commercially available from Senix Corporation of St. Bristol, Vt.

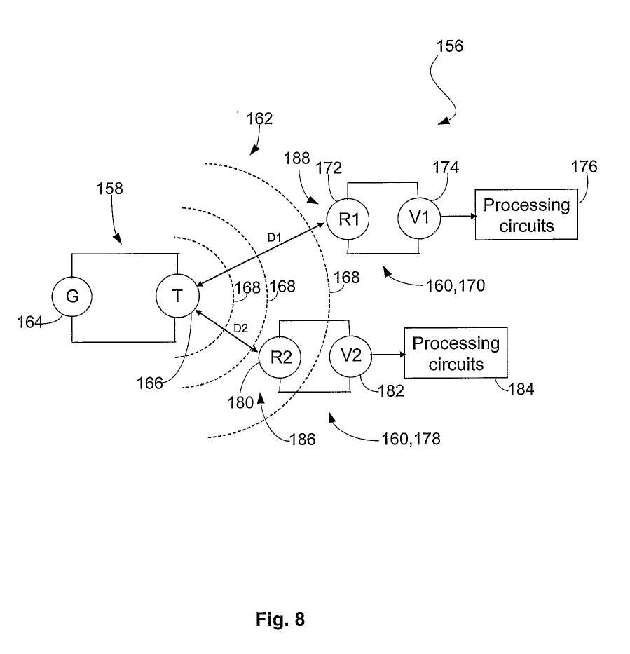

FIG. 8 is a block diagram that illustrates an embodiment system 156 where a plurality of distances, D1 and D2, between a transmitter 158 and a plurality of receivers 160, respectively, is calculated based on ultrasonic, or ultrasound, waves 162. The transmitter is configured to generate the ultrasonic waves, and the plurality of receivers is configured to detect the ultrasonic waves. The transmitter includes a generator G 164 and an ultrasonic transducer T 166, which is coupled to G. G drives T, which generates the ultrasonic waves. Wavefronts of the ultrasonic waves are shown as curved dashed lines 168 in FIG. 8.

In the example embodiment of FIG. 8, a first receiver 170 is located at distance D1 from T 166. The first receiver includes an ultrasonic transducer R1 172, front end amplifiers (not shown), buffers (not shown), voltage measurement circuits 174, and processing circuits 176, which are coupled to R1. The measured voltage V1 that results from the ultrasonic waves that are sensed at the first receiver can be used to calculate D1. An additional receiver, for example, a second receiver 178, which includes a second ultrasonic transducer R2 180, amplifiers (not shown), buffers (not shown), voltage measurement circuits 182, and processing circuits 184, can be positioned at a different location 186 from the location 188 of the first receiver. As shown in FIG. 8, R2 is located at distance D2 away from T.

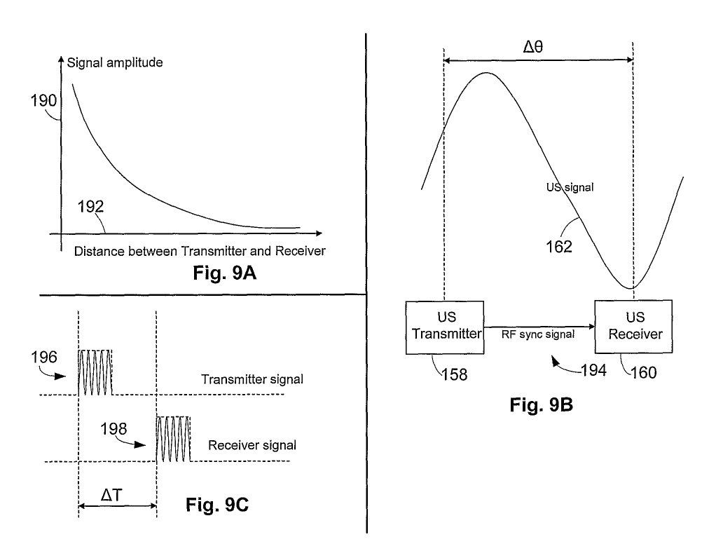

Distance calculations based on the transmission and receipt of ultrasonic waves 162 can be determined from measurements of the amplitude, phase, and/or the delay of the ultrasonic wave detected by the ultrasonic transducer 172 and 180. These different distance measurement possibilities using ultrasonic, or ultrasound, (“US”) signals are shown in FIGS. 9A-C. FIG. 9A shows the decrease in a US signal's amplitude 190 as a function of the distance 192 between a US transmitter 158 and a US receiver 160. The US signal's amplitude is inversely proportional to the distance between the transmitter and the receiver. By measuring the amplitude of the US signal, and comparing the measured amplitude to values in a calibration curve (not shown), it is possible to calculate distance between the US transmitter and the US receiver.

FIG. 9B shows the phase difference Δθ that can exist between a US transmitter 158 and a US receiver 160. By knowing Δθ and the wavelength of the US signal 162 it is possible to calculate the distance between the US transmitter and the US receiver. In embodiments of the present invention, the initial phase information, e.g., a synchronization signal 194, can be transmitted by the US transmitter via a radio frequency (“RF”) channel 78 to the US receiver. Since RF signals usually propagate 10⁶ times faster than ultrasonic signals, it can be assumed that the RF signal arrives at the US receiver with no delay, and thus, can provide synchronization between the US transmitter and the US receiver.

FIG. 9C shows the difference in the time between a US signal 196 output from a US transmitter 158 and the same US signal 198 received by a US receiver 160. In FIG. 9C, ΔT is the time difference between the time of transmission of the US signal by the US transmitter and the time of arrival of the US signal at the US receiver. By knowing ΔT and the propagation velocity of the US signal, it is possible to calculate the distance between the US transmitter and the US receiver. Synchronization between the US transmitter and the US receiver can be performed in a manner similar to the previously discussed synchronization method for the phase measurement technique.

### Data Acquisition:

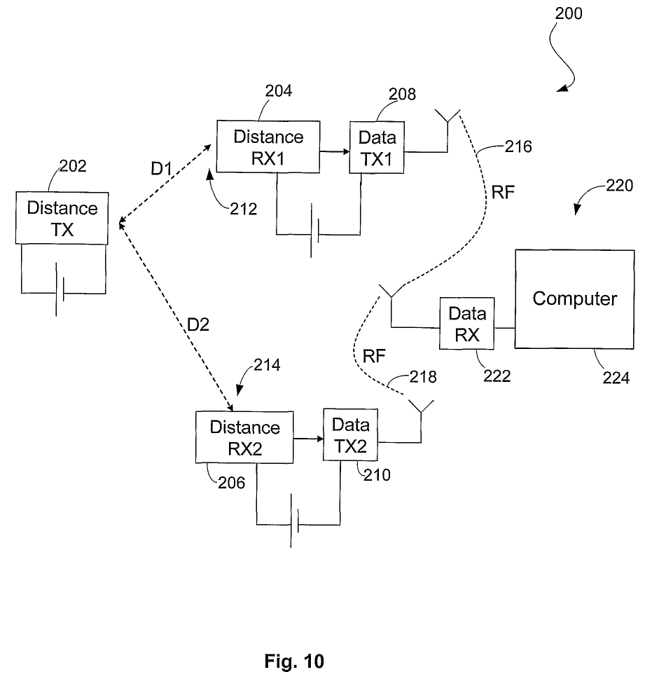

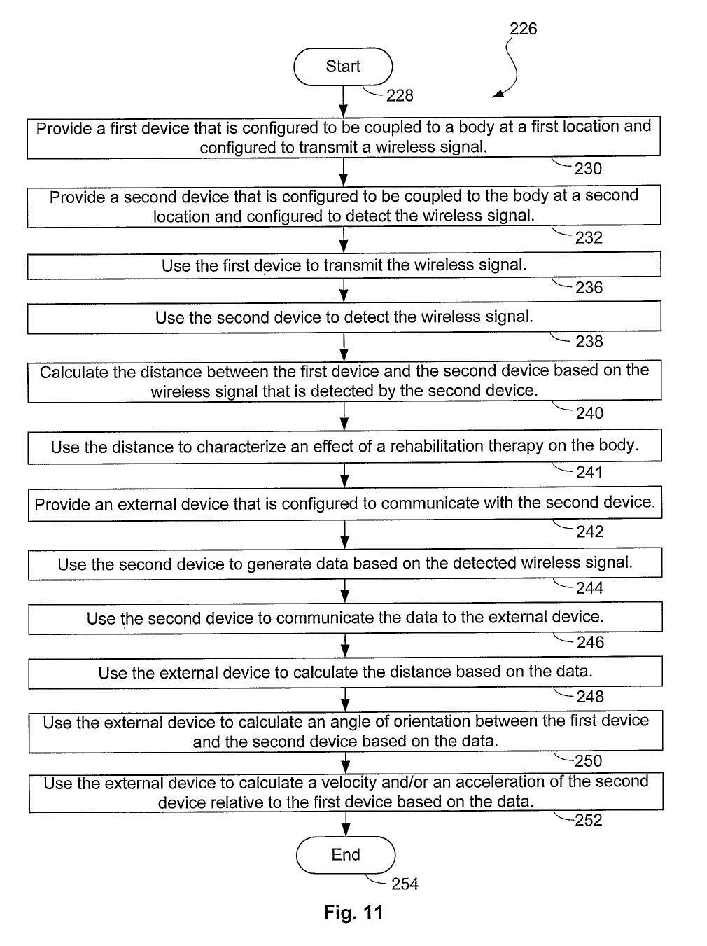

FIG. 10 is a schematic illustration of a data acquisition system 200 according to an embodiment of the present invention. In FIG. 10, a battery powered distance transmitter TX 202 generates a signal, e.g., a low-frequency magnetic field. The magnetic field generated by TX is detected and processed into data by a plurality of battery powered distance receivers RX1 204 and RX2 206, which are coupled to data transmitters TX1 208 and TX2 210, respectively, and positioned at different locations 212 and 214, respectively.

The data at RX1 204 and RX2 206 is transmitted through TX1 208 and TX2 210, respectively, via wireless RF links, or paths, 216 and 218, respectively, to an external device 220. The external device includes a data receiver Data RX 222, which is coupled to a computer 224. TX1, TX2, and Data RX can be off-the-shelf transceivers, e.g., the nRF2401A (a 2.4 GHz ultra low-power transceiver) or the nRF905 (a multi-band transceiver—operational at 433 MHz, 868 MHz, or 915 MHz), both of which are offered by Nordic Semiconductor of Norway.

After the data is received at Data RX 222, the data is communicated to the computer 224 where additional processing and/or calibration is performed on the data. Also, the computer is configured to calculate the distance D1 between RX1 204 and TX 202, and the distance D2 between RX2 206 and TX, based on the data. In addition, the computer is configured to display the resulting data, to control the data processing and/or calibration, and/or to control the other components, e.g., TX, RX1, RX2, TX1 208, TX2 210, and Data RX, of the system 200.

### Algorithm

An exemplary algorithm 226 that represents the steps taken by embodiment systems 10, 36, 67, 80, 82, 128, 156, and 200 is illustrated in FIG. 11. After the start 228 of the algorithm, in the next step 230, a first device 30 is provided, which is configured to be coupled to a body 14 at a first location 22, and configured to transmit a wireless signal 98, 126, and 162 (see FIGS. 6-8). Next, in step 232, a second device 28 is provided, which is configured to be coupled to the body at a second location 234 (see FIG. 1A), and configured to detect the wireless signal. At step 236, the first device is used to transmit the wireless signal. Next, at step 238, the second device is used to detect the wireless signal. At step 240, the distance D1 between the first and second devices is calculated based on the wireless signal that is detected by the second device. At step 241, the distance between the first and second devices is used to characterize an effect of a rehabilitation therapy on the body.

Next, at step 242, an external device 76 and 220 (see FIGS. 4 and 10) is provided, which is configured to communicate with the second device 28. At step 244, the second device is used to generate data based on the detected wireless signal 98, 126, and 162. Next, at step 246, the second device is used to communicate the data to the external device. At step 248, the external device is used to calculate the distance D1 based on the data. Next, at step 250, the external device is used to calculate an angle of orientation φ (see FIG. 6) between the first device 30 and the second device based on the data. At step 252, the external device is used to calculate the velocity and/or acceleration of the second device relative to the first device based on the data. The algorithm ends at step 254.

### Data Processing:

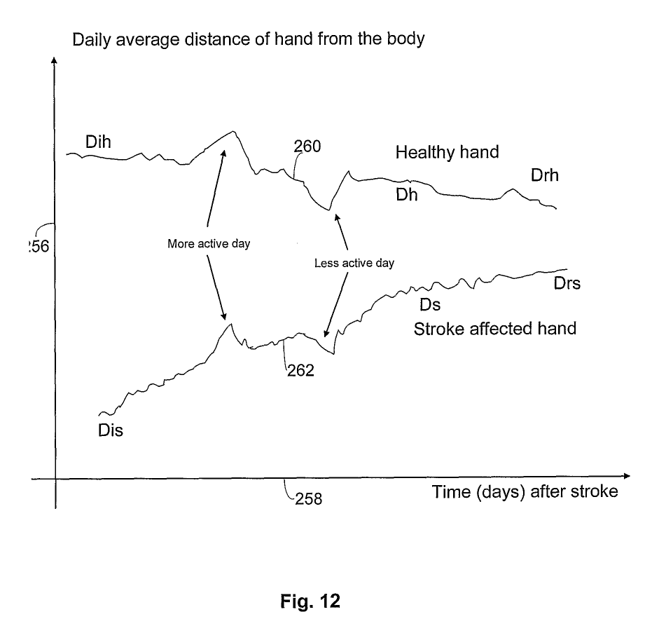

Distance and/or orientation data that is accumulated during a period of patient activity can be analyzed in real time or off-line by the external device 76 and 220, e.g., the computer 224. Different algorithms for processing data are available. For example, the average distance between a patient's hand 16 and body 14, i.e., torso 24, can be calculated and presented as shown in FIG. 12. FIG. 12 illustrates the change in the average movement distance 256 of a patient's hands relative to the torso over a period of time 258 after the patient 34 experiences a stroke. In particular, FIG. 12 includes a first trace 260 of the average distance of movement for the patient's healthy hand, and second trace 262 of the average distance of movement for the patient's impaired hand. Referring additionally to FIGS. 7, 8, and 10, the first and second traces can be calculated by the external device based on data from a first receiver 120, 170, and 204 that is coupled to the healthy hand and a second receiver 132, 178, and 206 that is coupled to the impaired hand. Referring additionally to FIGS. 1A-B and 2A-B, while the embodiments of FIGS. 1A-B and 2A-B only show devices 12 coupled to one of the patient's arms 26 and his or her torso, those having ordinary skill in the art should understand that the embodiments of the present invention can include devices coupled to both of the patient's arms as well as the patient's torso.

In FIG. 12, the first trace 260 includes the following three regions: Dih, which is an average initial post-stroke distance 256 of the healthy hand 16 from the patient's torso 24; Dh, which is an average daily distance of the patient's healthy hand from his or her torso during the rehabilitation process; and Drh, which is an average distance of the patient's healthy hand from his or her torso at the end of the rehabilitation process. Similarly, the second trace 262 includes the following three regions: Dis, which is an average initial post-stroke distance of the patient's stroke-affected hand from his or her torso; Ds, which is an average distance of the patients stroke-affected hand from his or her torso during the rehabilitation process; and Drs, which is the distance between the patient's stroke-affected hand and torso at the end of the rehabilitation process.

Accordingly, FIG. 12 shows the post-stroke recovery for the stroke-affected hand 16 in comparison to the healthy hand. Initially, in the Dih region of the first trace 260, the healthy hand is shown to compensate for the disability of the impaired hand, i.e., the healthy hand has a higher average distance 256 than during, or at the completion of, rehabilitation therapy. During rehabilitation therapy, the activity (average distance) of the patient's healthy hand from his or her torso 24 decreases, as shown in the Dh and Drh regions of the first trace. Daily activity can affect the average distance of both hands from the patient's torso. For example, walking or physical work can increase the average distance of the hands from the torso, while lower daily activity decreases the average distance of both hands from the torso.

In FIG. 12, the ratio Ds/Dis can be used as a rehabilitation indicator, which indicates improvement in mobility, e.g., the average distance 256, of the stroke-affected hand 16, as compared to the initial post-stroke condition. Another criterion that can be used is the ratio Ds/Dh, which is the ratio of the mobility for the stroke-affected hand and the healthy hand during rehabilitation therapy. It can be expected that the more complete the rehabilitation, the higher and more close to one Ds/Dh will become. Most likely, Ds/Dh will never be one because there is a difference between the left and rights hands, even in health subjects 34. Also, the ratio Dh/Dih can be considered as a rehabilitation indicator because it can indicate the effect of the rehabilitation on the patient's healthy hand. Finally, a combination of the above-mentioned ratios can be used as a rehabilitation indicator.

The calculated rehabilitation indicators may require normalization to compensate for the patient's daily activities, which may affect the position of the hand 16, but are not related to the rehabilitation, e.g., walking and performing physical work. This compensation can be performed by measuring the patient's general body activity, for example, by attaching accelerometers or pedometers to the patient's body 14. The calculated body activity is then used to change the resulting rehabilitation indicator values.

The following are additional examples of parameters that can be used to characterize limb mobility, for example, hand mobility in following discussion: an average of the distance 256 between the hand 16 and the torso 24 can be calculated for any time period, not necessarily for a 24 hour cycle; the standard deviation of the average distance between the hand and the torso, which is indicative of the actual hand movements and compensates for any static hand displacement; the number of movements of the hand away from the torso that exceed a pre-defined threshold of distance or angle; the number of hand movements per minute, hour, or day; speed parameters related to the movement of the hand, including, for example, average speed parameters and the standard deviation of speed parameters; acceleration parameters related to the movement of the hand, including, for example, average acceleration parameters and the standard deviation of acceleration parameters; and other kinetic and static parameters.

Referring again to FIG. 4, it should be noted that various additional devices 264 can be used to acquire parameters related to the movement of the limb 16-20 and 26. Example devices include the following: distance sensors, e.g., magnetic sensors and ultrasonic sensors; angle sensors, e.g., goniometers; acceleration and/or vibration sensors, e.g., micro-electro-mechanical systems (“MEMS”) acceleration sensor ADXL103 by Analog Devices of Norwood, Mass., or MEMS gyroscope ADIS16100 also by Analog Devices; and calculation of the limb and other body locations using a video camera and subsequent image processing, e.g., attaching a special marker to the limb, or special colored clothes in order to ease the computer recognition algorithms. A rehabilitation indicator can be based on some of the above-mentioned parameters, or their combination, and on additional kinetic/static parameters.

The foregoing detailed description of the present invention is provided for purposes of illustration, and it is not intended to be exhaustive or to limit the invention to the particular embodiments disclosed. The embodiments can provide different capabilities and benefits, depending on the configuration used to implement the key features of the invention. In particular, various types of distance, angle, position, and acceleration measurement devices, data channels, and data processing can be used in embodiments of the present invention. Also, referring again to FIGS. 1A, 2A, 4-8, and 10 the devices 12, e.g., the transmitters 30, 44, 68, 84, 116, 158, and 202 and receivers 28, 38-42, 70-74, 86, 120, 132, 170, 178, 204, and 206, that are used in the embodiments can be attached to, and/or implanted in, different parts of the body other than the hand 16, forearm 18, upper arm 20, and torso 24. Thus, the scope of the present invention is not limited to arm/hand rehabilitation assessment, and can be expanded to other parts of the body and to other applications beyond rehabilitation applications. In addition, while the previous discussion has focused on the use of the present invention to measure distance and orientation of various human body parts, the present invention can be used to measure the distance and orientation of parts of non-human bodies, e.g., animals other than humans. Accordingly, the scope of the invention is defined only by the following claims.

1. A system, comprising:
a first device configured to be coupled to a body at a first location and configured to generate a wireless signal; and
a second device configured to be coupled to the body at a second location, the second location being disposed apart from the first location by a distance;
the second device configured to detect the wireless signal and to generate data based on the detected wireless signal, the data generated being configured to be used to calculate the distance,
the distance being used to characterize the effect of the rehabilitation therapy on the body.

2. The system according to claim 1, wherein the distance is a first distance, the wireless signal of the first device is a first wireless signal, the first device configured to generate the first wireless signal at a first frequency, the system further comprising:
a third device configured to be coupled to the body at a third location, the third location being disposed apart from the second location by a second distance,
the third device configured to generate a second wireless signal at a second frequency,
the second device configured to detect the second wireless signal and to generate additional data based on the detected second wireless signal, the additional data generated being configured to be used to calculate the second distance,
the second distance being used to characterize the effect of the rehabilitation therapy on the body.

3. The system according to claim 2, wherein the third device is configured to generate the second wireless signal at the same time that the first device is configured to generate the first wireless signal.

4. The system according to claim 3, wherein the second device is configured to detect in a selectable manner a signal selected from the group consisting of the first wireless signal and the second wireless signal.

5. The system according to claim 1, further comprising:
the an external device configured to communicate with the second device,
the second device configured to communicate the data to the external device, and
the external device configured to calculate the distance based on the data.

6. The system according to claim 5, wherein the external device is configured to calculate an angle of orientation between the second device and the first device based on the data.

7. The system according to claim 5, wherein the external device is configured to communicate with the second device via a wireless communication path selected from the group consisting of a radio frequency path, an electrical current path through the body, a path configured for the communication of modulated sonic waves, a path configured for the communication of modulated ultrasonic waves, and an optical communication path.

8. The system according to claim 5, wherein the external device is configured to calculate a value selected from the group consisting of an average of the first distance over a period of time, a standard deviation of the first distance over a period of time, a number of times that the second device is moved relative to the first device over a period of time, a velocity of the second device relative to the first device, an average velocity of the second device relative to the first device over a period of time, an acceleration of the second device relative to the first device, and an average acceleration of the second device relative to the first device over a period of time based on the first distance.

9. The system according to claim 1, wherein at least one device of the first device and the second device is configured to be coupled to the body via a method selected from the group consisting of
implanting the at least one device within the body, and
attaching the at least one device to the body using a coupler selected from the group consisting of an adhesive, a piece of clothing, a strap, a belt, a clip, and a watch.

10. The system according to claim 1, wherein:
the first device is configured to be coupled to a torso of the body, and
the second device is configured to be coupled to any one of a hand or an arm.

11. The system according to claim 1, wherein the wireless signal is selected from the group consisting of a magnetic field, a low-frequency magnetic field, a sonic wave, and an ultrasonic wave.

12. The system according to claim 1, wherein at least one of the first device and the second device includes a component selected from the group consisting of a battery, a coil, orthogonal coils, a generator, a voltage measurement circuit, a transducer, a processing circuit, a transmitter, a receiver, and a transceiver.

13. The system according to claim 1, wherein at least one of the first device and the second device is a miniature stimulator.

14. The system according to claim 1, wherein at least one of the first device and the second device includes a transmitter and a receiver.

15. A system, comprising:
a transmitter configured to be coupled to a body at a first location and to transmit a wireless signal;
a plurality of receivers configured to be coupled to the body, each of the plurality of receivers configured to be coupled to the body at a different location disposed apart from the location of the transmitter by one of a plurality of distances, each of the plurality of receivers configured to detect the wireless signal and to generate data based on the detected wireless signal; and
an external device configured to communicate with the plurality of receivers and receive the generated data from the plurality of receivers,
the external device configured to calculate the plurality of distances between the plurality of receivers and the transmitter based on the data,
the plurality of distances being used to characterize the effect of the rehabilitation therapy on the body.

16. The system according to claim 15, wherein at least one device of the transmitter and the plurality of receivers is configured to be coupled to the body via a method selected from the group consisting of:
implanting the at least one device within the body, and
attaching the at least one device to the body using a coupler selected from the group consisting of an adhesive, a piece of clothing, a strap, a belt, a clip, and a watch.

17. The system according to claim 15, wherein the wireless signal is selected from the group consisting of a magnetic field, a low-frequency magnetic field, a sonic wave, and an ultrasonic wave.

18. The system according to claim 15, wherein:
the wireless signal is an ultrasonic wave; and
at least one receiver from the plurality of receivers is configured to generate the data based on at least one of an amplitude of the ultrasonic wave, a phase of the ultrasonic wave, and a time of propagation of the ultrasonic wave, the external device is configured to calculate the plurality of distances based on a characteristic of the ultrasonic wave detected by the plurality of receivers selected from the group consisting of an amplitude of the ultrasonic wave, a phase of the ultrasonic wave, and a time of propagation of the ultrasonic wave.

19. The system according to claim 15, wherein the external device is configured to calculate a plurality of angles of orientation between the plurality of receivers and the transmitter based on the data.

20. The system according to claim 19, further comprising an additional device selected from the group consisting of a distance sensor, an angle sensor, an acceleration sensor, a vibration sensor, and a video camera, wherein the additional device is coupled to the external device and configured to aid in the calculation of the plurality of distances and the plurality of angles of orientation.

21. The system according to claim 15, wherein the external device is configured to calculate, based on the data, a parameter selected from the group consisting of a velocity for each of the plurality of receivers relative to the transmitter, and an acceleration for each of the plurality of receivers relative to the transmitter.

22. The system according to claim 15, wherein:
one of the plurality of receivers is configured to be coupled to a healthy limb of the body;
another of the plurality of receivers is configured to be coupled to an impaired limb of the body; and
the external device is configured to compare the distance between the one of the plurality of receivers and the transmitter to the distance between the another of the plurality of receivers and the transmitter.

23. A method comprising:
coupling a first device to the body at a first location;
coupling a second device at a second location, the second location spaced apart from the first location;
transmitting a wireless signal from the first device;
receiving the wireless signal;
calculating a distance between the first device and the second device based on the wireless signal; and
characterizing the effect of the rehabilitation therapy on the body based on the distance.

24. The method according to claim 23, further comprising:
generating a data based on the wireless signal, the calculating performed based on the data.

25. The method according to claim 24, further comprising:
calculating an angle of orientation between the first device and the second device based on the data.

26. The method according to claim 24, further comprising:
calculating, based on the data, a parameter selected from the group consisting of a velocity of the second device relative to the first device, and an acceleration of the second device relative to the first device.

27. The method according to claim 23, wherein:
the receiving the wireless signal is performed by the second device; and
the calculating a distance between the first device and the second device based on the wireless signal is performed by an external device.

28. A system comprising:
a plurality of transmitters configured to be coupled to a body, each of the plurality of transmitters configured to transmit a wireless signal at a unique frequency;
a receiver configured to be coupled to the body, the receiver being spaced apart from each of the plurality of transmitters by a plurality of distances, each of the plurality of transmitters being spaced apart from each other by one of a plurality of distances, the receiver configured to receive each of the wireless signals from each of the plurality of transmitters and generate data based on the detected wireless signals; and
an external device configured to receive the data from the receiver, the external device configured to calculate the plurality of distances between the receiver and the plurality of transmitters based on the data, the plurality of distances being used to characterize the effect of the rehabilitation therapy on the body.

29. The system according to claim 28, wherein the receiver is configured to selectively receive each of the wireless signals from the plurality of transmitters.
